SEQUENCE LISTING (8 sequences)

SEQ ID NO: 1 - DNA, 2229 bp, Danio rerio
  CDS (1)..(2229)
atgatgacgtttctttgcacacggtctggtcgccgtgctagtggtgtggagtgcagaatcgccgctgaacgcgcttactttcgagtgcgtggactcccggttgccaatatgattggctggtggccacgcctctgcccagtgatgtcactggcactgctgtgggcgtgttcagtgggggcgggttcagactgcaaatctgtggccattgagactgacagccgcatagcagaacaaacacagcagcgtcacctacaggctctgttcgacaagtatggccagaacggcagcatctccctagaaggcctcttcaacctacttaaaggggtcgggcttgaccgcatccggaaagtgatggtgcatcatcctggaaatgcccataatcacacacacacgcatgatcacacacacactcatgtggacaaactcacggcgcacacacatccggtcaccaccaagaagggagacatggatcacagcgtggagaagagtgaccctgtcccaaaagcacagccagatcctgcctctgggaagaaaagccagtcagatgcgcatcacaacctgtacatgaagatgaaccaggaatccaccacagctttgactacgccgtcatatgttaccagatcacggcggaccaatcgcagcgccgattatgattttacacaggaccacgcctcctttagccccagtcagcccaatgtgacacactcaaaccacacccatcatgatgaggacacgcccacacaccagcatgatgaccatgatgagcacgaacatgcccgtgctagtttagggtgtcaaaatgcctccacgatcctgcagacgcatggcatgagaaaggaagcaagtctctcagttaaggacttcagtttcctctgccctgctcttctcatgcagattgattccaagtcttgcatcgtgcatgaagacgaggacgagcattcagatcattcccatcatcacaaacaccaccaccatcatcatgatcaccaacacctgcagcatccacataaccacaccaatggaagaggccagaggaacactccagtctacatcgcatggcttggagggtttctctccatcactctgatcagtttgctggcgttggttggtgtggttttgatcccactcatgaacagagtttgcttcaacttcctgctgagcttcctggtggcccttgcagtgggcactctgagcggagacgctctcctccacctcataccacattctcagggtcatcaccatcacggccactctgaagagcacgctgaagaggaggactcccttcgccctgtgtggaccggactcacagctctaagtggagtttacatcatgttcctcatcgaacacttcctgacccttggcaaaatgtacaaagacaaaaaccagaaggtgcagaagagggttgatctcaccacagaagttttggagtctgagaaactgccatcattagaagaaaatgatgtcaaaattgaagctgctgaaacgaatggtgggcgtgcactggcagaggaggaggaggtgatgttgggggcggagctctacaacgacatagactgcgagaacaaatgccactcccacttccatgacacggtcggccaatcggatgagcagcatcatcatcatcacgactaccaccacatactgcatcatcaccactcccagaaccaccacccgcacacacacacgcacagacacacacactcctactcgcagcagcactttgagcaggctggtgtggccacactcgcctggatggtcatcatgggagacggactgcacaacttcagtgatggacttgccataggggcggctttcacagaaggtttgtccagtggtcttagtacctcagtcgctgtgttctgccatgagcttcctcatgaactcggtgattttgccgtcctactgaaagccggtatgtcagttcgacaggccatgctgtataatctgctgtcagcactgatgggatatctgggcatgatcatcgggattctcatcggacattatgctgaaaatgttgccacatggatctttgctctcacagctgggttattcatgtacgtcgcgctcgtggacatggtacctgagatgctgcacaatgacgcgagcgaagcaggtttcagtcactacggcttcttcctcctgcagaacgctgggatactcctaggcttcggcatcatgcttatcattgctgtctttgaggacaggatccaactggacttaggttactga

SEQ ID NO: 2 - protein, 742 aa, Danio rerio
MMTFLCTRSGRRASGVECRIAAERAYFRVRGLPVANMIGWWPRLCPVMSLALLWACSVGAGSDCKSVAIETDSRIAEQTQQRHLQALFDKYGQNGSISLEGLFNLLKGVGLDRIRKVMVHHPGNAHNHTHTHDHTHTHVDKLTAHTHPVTTKKGDMDHSVEKSDPVPKAQPDPASGKKSQSDAHHNLYMKMNQESTTALTTPSYVTRSRRTNRSADYDFTQDHASFSPSQPNVTHSNHTHHDEDTPTHQHDDHDEHEHARASLGCQNASTILQTHGMRKEASLSVKDFSFLCPALLMQIDSKSCIVHEDEDEHSDHSHHHKHHHHHHDHQHLQHPHNHTNGRGQRNTPVYIAWLGGFLSITLISLLALVGVVLIPLMNRVCFNFLLSFLVALAVGTLSGDALLHLIPHSQGHHHHGHSEEHAEEEDSLRPVWTGLTALSGVYIMFLIEHFLTLGKMYKDKNQKVQKRVDLTTEVLESEKLPSLEENDVKIEAAETNGGRALAEEEEVMLGAELYNDIDCENKCHSHFHDTVGQSDEQHHHHHDYHHILHHHHSQNHHPHTHTHRHTHSYSQQHFEQAGVATLAWMVIMGDGLHNFSDGLAIGAAFTEGLSSGLSTSVAVFCHELPHELGDFAVLLKAGMSVRQAMLYNLLSALMGYLGMIIGILIGHYAENVATWIFALTAGLFMYVALVDMVPEMLHNDASEAGFSHYGFFLLQNAGILLGFGIMLIIAVFEDRIQLDLGY

SEQ ID NO: 3 - DNA, 2268 bp, Homo sapiens
  CDS (1)..(2268)
atggcgaggaagttatctgtaatcttgatcctgacctttgccctctctgtcacaaatccccttcatgaactaaaagcagctgctttcccccagaccactgagaaaattagtccgaattgggaatctggcattaatgttgacttggcaatttccacacggcaatatcatctacaacagcttttctaccgctatggagaaaataattctttgtcagttgaagggttcagaaaattacttcaaaatataggcatagataagattaaaagaatccatatacaccatgaccacgaccatcactcagaccacgagcatcactcagaccatgagcgtcactcagaccatgagcatcactcagaccacgagcatcactctgaccatgatcatcactctcaccataatcatgctgcttctggtaaaaataagcgaaaagctctttgcccagaccatgactcagatagttcaggtaaagatcctagaaacagccaggggaaaggagctcaccgaccagaacatgccagtggtagaaggaatgtcaaggacagtgttagtgctagtgaagtgacctcaactgtgtacaacactgtctctgaaggaactcactttctagagacaatagagactccaagacctggaaaactcttccccaaagatgtaagcagctccactccacccagtgtcacatcaaagagccgggtgagccggctggctggtaggaaaacaaatgaatctgtgagtgagccccgaaaaggctttatgtattccagaaacacaaatgaaaatcctcaggagtgtttcaatgcatcaaagctactgacatctcatggcatgggcatccaggttccgctgaatgcaacagagttcaactatctctgtccagccatcatcaaccaaattgatgctagatcttgtctgattcatacaagtgaaaagaaggctgaaatccctccaaagacctattcattacaaatagcctgggttggtggttttatagccatttccatcatcagtttcctgtctctgctgggggttatcttagtgcctctcatgaatcgggtgtttttcaaatttctcctgagtttccttgtggcactggccgttgggactttgagtggtgatgcttttttacaccttcttccacattctcatgcaagtcaccaccatagtcatagccatgaagaaccagcaatggaaatgaaaagaggaccacttttcagtcatctgtcttctcaaaacatagaagaaagtgcctattttgattccacgtggaagggtctaacagctctaggaggcctgtatttcatgtttcttgttgaacatgtcctcacattgatcaaacaatttaaagataagaagaaaaagaatcagaagaaacctgaaaatgatgatgatgtggagattaagaagcagttgtccaagtatgaatctcaactttcaacaaatgaggagaaagtagatacagatgatcgaactgaaggctatttacgagcagactcacaagagccctcccactttgattctcagcagcctgcagtcttggaagaagaagaggtcatgatagctcatgctcatccacaggaagtctacaatgaatatgtacccagagggtgcaagaataaatgccattcacatttccacgatacactcggccagtcagacgatctcattcaccaccatcatgactaccatcatattctccatcatcaccaccaccaaaaccaccatcctcacagtcacagccagcgctactctcgggaggagctgaaagatgccggcgtcgccactttggcctggatggtgataatgggtgatggcctgcacaatttcagcgatggcctagcaattggtgctgcttttactgaaggcttatcaagtggtttaagtacttctgttgctgtgttctgtcatgagttgcctcatgaattaggtgactttgctgttctactaaaggctggcatgaccgttaagcaggctgtcctttataatgcattgtcagccatgctggcgtatcttggaatggcaacaggaattttcattggtcattatgctgaaaatgtttctatgtggatatttgcacttactgctggcttattcatgtatgttgctctggttgatatggtacctgaaatgctgcacaatgatgctagtgaccatggatgtagccgctgggggtatttctttttacagaatgctgggatgcttttgggttttggaattatgttacttatttccatatttgaacataaaatcgtgtttcgtataaatttctag

SEQ ID NO: 4 - protein, 755 aa, Homo sapiens
MARKLSVILILTFALSVTNPLHELKAAAFPQTTEKISPNWESGINVDLAISTRQYHLQQLFYRYGENNSLSVEGFRKLLQNIGIDKIKRIHIHHDHDHHSDHEHHSDHERHSDHEHHSDHEHHSDHDHHSHHNHAASGKNKRKALCPDHDSDSSGKDPRNSQGKGAHRPEHASGRRNVKDSVSASEVTSTVYNTVSEGTHFLETIETPRPGKLFPKDVSSSTPPSVTSKSRVSRLAGRKTNESVSEPRKGFMYSRNTNENPQECFNASKLLTSHGMGIQVPLNATEFNYLCPAIINQIDARSCLIHTSEKKAEIPPKTYSLQIAWVGGFIAISIISFLSLLGVILVPLMNRVFFKFLLSFLVALAVGTLSGDAFLHLLPHSHASHHHSHSHEEPAMEMKRGPLFSHLSSQNIEESAYFDSTWKGLTALGGLYFMFLVEHVLTLIKQFKDKKKKNQKKPENDDDVEIKKQLSKYESQLSTNEEKVDTDDRTEGYLRADSQEPSHFDSQQPAVLEEEEVMIAHAHPQEVYNEYVPRGCKNKCHSHFHDTLGQSDDLIHHHHDYHHILHHHHHQNHHPHSHSQRYSREELKDAGVATLAWMVIMGDGLHNFSDGLAIGAAFTEGLSSGLSTSVAVFCHELPHELGDFAVLLKAGMTVKQAVLYNALSAMLAYLGMATGIFIGHYAENVSMWIFALTAGLFMYVALVDMVPEMLHNDASDHGCSRWGYFFLQNAGMLLGFGIMLLISIFEHKIVFRINF

SEQ ID NO: 5 - DNA, 2298 bp, Mus musculus
  CDS (1)..(2298)
atggccacagatttatctgtaatcatgatcttgacctttgccctttgggttacaagcccccttcatgaactacaatcaacagctgctttctctcagactactgagaaaataaattcaaattgggaacctggtgttaatgttgacttggcagttaccatgcagcgacaccatctgcagcagctattctaccgctacggagagaatgattccttgtctgttgaaggcttcagaaaattgcttcagaacataggcatagataagattaaaagagtccatatacaccatgaccacgagcatcatgctgaccacgagcatcactcggaccatgagcatcactcggaccacgagcatcactcggaccacgagcatcactcggaccacgagcatcactcggaccacgagcaccattcccaccgcagtcacacggttgctggtaaaaacaatcggaaagccttttgtccagaccttgactctgataattcaggtaaaaatcctagaactagtctagggaaaggatctcgcccagcagagcacatgaatggtaggaggaacatcaaggagagtgcaagctctagtgaagtgacctcggcggtatacaacgctgtctctgaaggaactcgctttgtagagacaatagagactccaaaacctgggagacgcaccaaagatgtaaacccttctaccccacccagcatcacggagaaaagccgagtgggccggctgagtcggctagctaggaagaaaagcaatgagtctgtgagtgagcccagaaagagctttatgtattccagaaacacaaatgacaatattcaggagtgtttcaatacaaccaagctgctgacatcccatggcatgagcatccaggctctgttgaatgcaacggaatttaactatctctgcccagccatcatcaatcaaattgatgctcgggcttgtctgattcatacagcaagtgagaagaaggcagaaatccctccaaagacctattctttacaaatagcctggcttggtggcttcatagccatttccatcatcagtttcctgtctctgctgggagtcatcttggtgccactcatgaaccgggtatttttcaagttcctgctgagcttcctcgtggcgctggccgtcggaacgctgagtggcgatgctctgttacatcttctcccacactctcatgcaagtcatcagcacagtcatagccatgaagagccagcgatggaaatgaaaagaggccccctgttcagccacctgtcggctcagaatatagaagaaagctcctattttgattccacgtggaaaggtctgacggctctagggggcttatatttcatgtttcttgtggaacacgtactcacactgatcaagcaatttaaagataagaaaaagaagaatcaaaagaaacctgaaaatgatgaggatgtggagagcaagaagcagctgtccaaatacgactctcagctttcctcaaatgaagagaaggtggacccaggggaacgacctgaaagctatctgcgagccgactcccaagagccctccccctttgattcccagcagccgacgatgttggaagaggaagaggtcatgatagcccatgcacacccacaagaagtctacaatgaatatgtgcccaggggctgcaagaacaagtgccattcacacttccacgatacgctgggccagtccgacgacctcatccaccaccatcacgactaccatcacattctgcaccaccaccaccaccagaaccaccaccctcacagccacagccagcgctactctcgagaggagctgaaggacgccggcattgccacattggcctggatggtgatcatgggcgacgggctgcacaatttcagtgacggccttgctattggtgctgccttcaccgagggtttgtccagtgggctaagcacctctgtcgctgtgttctgtcatgaactgcctcatgaactaggtgactttgctgttttgctaaaggctggcatgactgtcaagcaggctgtgctctataatgctctgtcagccatgttggcctaccttggaatggcaacagggatattcatcgggcattatgcagaaaatgtttctatgtggatattcgcactcactgccggcttgttcatgtatgtcgctctggttgacatggtgcctgagatgttgcacaatgatgctagtgatcacggatgcagccgttggggatatttcttcctgcagaatgctgggatacttctcggttttggaattatgttactcatttccatatttgagcataaaattgtgtttcgtataaatttctaa

SEQ ID NO: 6 - protein, 765 aa, Mus musculus
MATDLSVIMILTFALWVTSPLHELQSTAAFSQTTEKINSNWEPGVNVDLAVTMQRHHLQQLFYRYGENDSLSVEGFRKLLQNIGIDKIKRVHIHHDHEHHADHEHHSDHEHHSDHEHHSDHEHHSDHEHHSDHEHHSHRSHTVAGKNNRKAFCPDLDSDNSGKNPRTSLGKGSRPAEHMNGRRNIKESASSSEVTSAVYNAVSEGTRFVETIETPKPGRRTKDVNPSTPPSITEKSRVGRLSRLARKKSNESVSEPRKSFMYSRNTNDNIQECFNTTKLLTSHGMSIQALLNATEFNYLCPAIINQIDARACLIHTASEKKAEIPPKTYSLQIAWLGGFIAISIISFLSLLGVILVPLMNRVFFKFLLSFLVALAVGTLSGDALLHLLPHSHASHQHSHSHEEPAMEMKRGPLFSHLSAQNIEESSYFDSTWKGLTALGGLYFMFLVEHVLTLIKQFKDKKKKNQKKPENDEDVESKKQLSKYDSQLSSNEEKVDPGERPESYLRADSQEPSPFDSQQPTMLEEEEVMIAHAHPQEVYNEYVPRGCKNKCHSHFHDTLGQSDDLIHHHHDYHHILHHHHHQNHHPHSHSQRYSREELKDAGIATLAWMVIMGDGLHNFSDGLAIGAAFTEGLSSGLSTSVAVFCHELPHELGDFAVLLKAGMTVKQAVLYNALSAMLAYLGMATGIFIGHYAENVSMWIFALTAGLFMYVALVDMVPEMLHNDASDHGCSRWGYFFLQNAGILLGFGIMLLISIFEHKIVFRINF

SEQ ID NO: 7 - DNA, 25 bp, Artificial
  antisense
cggaaacagcgcgagtgtcttttgt

SEQ ID NO: 8 - DNA, 25 bp, Artificial
  antisense
accgtgtgcaaagaaacgtcatcat

(19) United States
(12) Patent Application Publication
(10) Pub. No.: US 2009/0203749 A1
(43) Pub. Date: Aug. 13, 2009

(54) AGENT FOR CONTROL OF DEGRANULATION REACTION AND CYTOKINE PRODUCTION

(75) Inventors: Toshio Hirano, Kanagawa (JP); Keigo Nishida, Kanagawa (JP)

Correspondence Address: LEYDIG VOIT & MAYER, LTD, TWO PRUDENTIAL PLAZA, SUITE 4900, 180 NORTH STETSON AVENUE, CHICAGO, IL, 60601-6731, US

(73) Assignee: RIKEN, Wako-shi, Saitama (JP)

(21) Appl. No.: 11/815,187

(22) PCT Filed: Jan. 31, 2006
(86) PCT No.: PCT/JP2006/301932

§ 371 Date: Sep. 11, 2007

(30) Foreign Application Priority Data
Jan. 31, 2005 (JP) 2005-023196

(51) Int. Cl. A61K 31/44; C07D 213/02; C12Q 1/02

(52) U.S. Cl. 514/357; 546/329; 435/29

(57) ABSTRACT

To provide an agent capable of controlling the degranulation reaction and/or cytokine production of mast cell and the like. The present invention provides an agent for controlling a degranulation reaction and an agent for controlling a cytokine production, comprising a substance capable of controlling an intracellular zinc ion concentration, particularly, a zinc ion, a zinc ion chelator, an agent for controlling the expression and/or function of a zinc ion-requiring protein, or an agent for controlling the expression and/or function of a zinc ion transporter, as an active ingredient.

### TECHNICAL FIELD

The present invention relates to an agent capable of controlling degranulation reaction and cytokine production, and especially relates to an agent capable of treating allergic diseases and various inflammatory diseases, in which cells that can release chemical mediators and/or cells with an ability to produce cytokines (for example, a mast cell) are involved, by controlling degranulation reaction and/or cytokine production in the cells.

### BACKGROUND ART

Current anti-allergic agents include antihistamines, steroids with an immunosuppressive effect, and the like. The antihistamine suppresses the association of histamine released from effector cells such as mast cells and a specific receptor thereof expressed on target cells. However, the chemical mediators released from the effector cells are not limited to histamine, but include serotonin, prostaglandin etc. The antihistamine cannot be expected to have any effect on these chemical mediators. On the other hand, it is known that although the steroid has strong immunosuppressive and anti-inflammatory activities and is used frequently as an anti-allergic drug, side effects such as opportunistic infection are often observed.

It has been revealed that the mast cell has granules containing chemical mediators such as histamine and cytokines, and that the degranulation reaction in the mast cell plays an important role in allergic diseases such as pollinosis, bronchial asthma and atopic dermatitis, and various inflammatory diseases including autoimmune diseases. Therefore, suppression of the degranulation reaction is effective as a treatment method of these diseases.

Agents that suppress the release of the chemical mediators due to the degranulation reaction by stabilizing the membrane of the mast cell include Intal (sodium cromoglycate), Rizaben (tranist) and the like, and they have already been used as anti-allergic agents in clinical application. The degranulation is caused by signal transduction through IgE receptor. Since this process depends on calcium ion, the control of the degranulation reaction has also been attempted by controlling the intracellular calcium concentration. However, as the calcium ion plays an important role in any situation in the body, to control the intracellular calcium concentration may have an effect on other biological functions.

For conventional techniques regarding the degranulation reaction and the anti-allergic agents, one can see Turner H. & Kinet J P. Signalling through the high-affinity IgE receptor Fc epsilon R1. Nature 402, B24-30 (1999), and Blank U, Rivera J. The ins and outs of IgE-dependent mast-cell exocytosis. TRENDS in Immunology 25, 266-273 (2004). For conventional techniques regarding the cytokine production from mast cells, one can see Burd P R, Rogers H W, Gordon L R, Martin C A, Jayaraman S, Wilson S D, Dvorak A M, Galli S J, Dorf M E. Interleukin 3-dependent and -independent mast cells stimulated with IgE and antigen express multiple cytokines. J. Exp. Med. 170, 240-257 (1989), and Song J S, Haleem-Smith H, Arudchandran R, Gomez J, Scott P M, Mill J F, Tan T H, Rivera J. Tyrosine phosphorylation of Vav stimulates IL-6 production in mast cells by a Rac/c-Jun N-terminal kinase-dependent pathway. J. Immunology. 163, 802-810 (1999).

### DISCLOSURE OF INVENTION

An object of the present invention is to provide novel agents controlling the degranulation reaction and cytokine production, and particularly, to provide agents that can control the release of chemical mediators such as histamine and/or agents that can control the production of cytokines from cells such as mast cell. In addition, an object of the present invention is to provide an application of the agents controlling the degranulation reaction and/or the agents controlling the cytokine production to allergic diseases and inflammatory diseases.

In the course of solving the problems, the present inventors have found by keen examination that the degranulation reaction and cytokine production can be controlled by controlling the intracellular zinc ion concentration. Further, the present inventors have led to the completion of the invention by confirming that the control of the degranulation reaction by controlling the zinc ion concentration is reversible. That is, the present invention is as following.

[1] An agent for controlling a degranulation reaction, which comprises a substance capable of controlling an intracellular zinc ion concentration as an active ingredient.
[2] The agent for controlling the degranulation reaction described in [1] above, wherein the substance capable of controlling the zinc ion concentration is a zinc ion.
[3] The agent for controlling the degranulation reaction described in [2] above, wherein the zinc ion is introduced into a cell through a zinc ionophore.
[4] The agent for controlling the degranulation reaction described in [1] above, wherein the substance capable of controlling the zinc ion concentration is a zinc ion chelator.
[5] The agent for controlling the degranulation reaction described in [4] above, wherein the zinc ion chelator is at least one selected from the group consisting of 2,3-dimercapto-1-propanesulfonic acid (DMPS), N,N,N′,N′-tetrakis(2-pyridylmethyl)ethylenediamine (TPEN), ethylenediamine (EDTA), and N-(6-methoxy-8-quinolyl)-p-toluenesulfonamide (TSQ).
[6] The agent for controlling the degranulation reaction described in [1] above, wherein the substance capable of controlling the zinc ion concentration is a substance regulating the expression and/or function of a zinc ion-requiring protein.
[7] The agent for controlling the degranulation reaction described in [6] above, wherein the zinc ion-requiring protein is at least one selected from the group consisting of Raf-1, PKCα, GEF-H1, HDAC, SQSTM1, ubiquitin-protein ligase E3, ubiquitin-conjugating enzyme E2, metallothionein, phosphatase, Snail, TRAF6, Paxillin, Zyxin and Jade-1.
[8] The agent for controlling the degranulation reaction described in [1] above, wherein the substance capable of controlling the zinc ion concentration is a substance regulating the expression and/or function of a zinc ion transporter.
[9] The agent for controlling the degranulation reaction described in [8] above, wherein the zinc ion transporter is at least one selected from the group consisting of human ZIPs including LIV family and human CDFs.
[10] The agent for controlling the degranulation reaction described in any one of [1] to [9] above, wherein the cell is at least one selected from the group consisting of a neutrophil, an eosinophil, a basophil, a mast cell, a natural killer cell, a natural killer T cell, a cytotoxic T cell and a platelet.
[11] The agent for controlling the degranulation reaction described in [10] above, wherein the cell is a mast cell.
[12] An agent for controlling a cytokine production, which comprises a substance capable of controlling an intracellular zinc ion concentration as an active ingredient.
[13] The agent for controlling the cytokine production described in [12] above, wherein the substance capable of controlling the zinc ion concentration is a zinc ion.
[14] The agent for controlling the cytokine production described in [13] above, wherein the zinc ion is introduced into a cell through a zinc ionophore.
[15] The agent for controlling the cytokine production described in [12] above, wherein the substance capable of controlling the zinc ion concentration is a zinc ion chelator.
[16] The agent for controlling the cytokine production described in [15] above, wherein the zinc ion chelator is at least one selected from the group consisting of 2,3-dimercapto-1-propanesulfonic acid (DMPS), N,N,N′,N′-tetrakis(2-pyridylmethyl)ethylenediamine (TPEN), ethylenediamine (EDTA), and N-(6-methoxy-8-quinolyl)-p-toluenesulfonamide (TSQ).
[17] The agent for controlling the cytokine production described in [12] above, wherein the substance capable of controlling the zinc ion concentration is a substance regulating the expression and/or function of a zinc ion-requiring protein.
[18] The agent for controlling the cytokine production described in [17] above, wherein the zinc ion-requiring protein is at least one selected from the group consisting of Raf-1, PKCα, GEF-H1, HDAC, SQSTM1, ubiquitin-protein ligase E3, ubiquitin-conjugating enzyme E2, metallothionein, phosphatase, Snail, TRAF6, Paxillin, Zyxin and Jade-1.
[19] The agent for controlling the cytokine production described in [12] above, wherein the substance capable of controlling the zinc ion concentration is a substance regulating the expression and/or function of a zinc ion transporter.
[20] The agent for controlling the cytokine production described in [19] above, wherein the zinc ion transporter is at least one selected from the group consisting of human ZIPs including LIV family and human CDFs.
[21] The agent for controlling the cytokine production described in any one of [12] to [20] above, wherein the cell is at least one selected from the group consisting of a neutrophil, an eosinophil, a basophil, a mast cell, a natural killer cell, a natural killer T cell, a cytotoxic T cell and a platelet.
[22] The agent for controlling the cytokine production described in [21] above, wherein the cell is a mast cell.
[23] A prophylactic and/or therapeutic drug for an allergic disease and/or an inflammatory disease including an autoimmune disease, which comprises the agent for controlling the degranulation reaction described in any one of [1] to [11] above or the agent for controlling the cytokine production described in any one of [12] to [22] above.
[24] A prophylactic and/or therapeutic drug for an allergic disease and/or an inflammatory disease including an autoimmune disease, which comprises a substance capable of controlling an intracellular zinc ion concentration as an active ingredient.
[25] The prophylactic and/or therapeutic drug described in [24] above, wherein the substance capable of controlling the zinc ion concentration is a zinc ion.
[26] The prophylactic and/or therapeutic drug described in [25] above, wherein the zinc ion is introduced into a cell through a zinc ionophore.
[27] The prophylactic and/or therapeutic drug described in [24] above, wherein the substance capable of controlling the zinc ion concentration is a zinc ion chelator.
[28] The prophylactic and/or therapeutic drug described in [27] above, wherein the zinc ion chelator is at least one selected from the group consisting of 2,3-dimercapto-1-propanesulfonic acid (DMPS), N,N,N′,N′-tetrakis(2-pyridylmethyl)ethylenediamine (TPEN), ethylenediamine (EDTA), and N-(6-methoxy-8-quinolyl)-p-toluenesulfonamide (TSQ).
[29] The prophylactic and/or therapeutic drug described in [24] above, wherein the substance capable of controlling the zinc ion concentration is a substance regulating the expression and/or function of a zinc ion-requiring protein.
[30] The prophylactic and/or therapeutic drug described in [29] above, wherein the zinc ion-requiring protein is at least one selected from the group consisting of Raf-1, PKCα, GEF-H1, HDAC, SQSTM1, ubiquitin-protein ligase E3, ubiquitin-conjugating enzyme E2, metallothionein, phosphatase, Snail, TRAF6, Paxillin, Zyxin and Jade-1.
[31] The prophylactic and/or therapeutic drug described in [24] above, wherein the substance capable of controlling the zinc ion concentration is a substance regulating the expression and/or function of a zinc ion transporter.
[32] The prophylactic and/or therapeutic drug described in [31] above, wherein the zinc ion transporter is at least one selected from the group consisting of human ZIPs including LIV family and human CDFs.
[33] The prophylactic and/or therapeutic drug described in any one of [24] to [32] above, wherein the cell is at least one selected from the group consisting of a neutrophil, an eosinophil, a basophil, a mast cell, a natural killer cell, a natural killer T cell, a cytotoxic T cell and a platelet.
[34] The prophylactic and/or therapeutic drug described in [33] above, wherein the cell is a mast cell.
[35] A method of controlling the degranulation reaction, which comprises controlling an intracellular zinc ion concentration in vitro.
[36] The method described in [35] above, wherein the zinc ion concentration is controlled by a zinc ion.
[37] The method described in [36] above, wherein the zinc ion is introduced into a cell through a zinc ionophore.
[38] The method described in [35] above, wherein the zinc ion concentration is controlled by a zinc ion chelator.
[39] The method described in [38] above, wherein the zinc ion chelator is at least one selected from the group consisting of 2,3-dimercapto-1-propanesulfonic acid (DMPS), N,N,N′,N′-tetrakis(2-pyridylmethyl)ethylenediamine (TPEN), ethylenediamine (EDTA), and N-(6-methoxy-8-quinolyl)-p-toluenesulfonamide (TSQ).
[40] The method described in [35] above, wherein the zinc ion concentration is controlled by regulating the expression and/or function of a zinc ion-requiring protein.
[41] The method described in [40] above, wherein the zinc ion-requiring protein is at least one selected from the group consisting of Raf-1, PKCα, GEF-H1, HDAC, SQSTM1, ubiquitin-protein ligase E3, ubiquitin-conjugating enzyme E2, metallothionein, phosphatase, Snail, TRAF6, Paxillin, Zyxin and Jade-1.
[42] The method described in [35] above, wherein the zinc ion concentration is controlled by regulating the expression and/or function of a zinc ion transporter.
[43] The method described in [42] above, wherein the zinc ion transporter is at least one selected from the group consisting of human ZIPs including LIV family and human CDFs.
[44] The method described in any one of [35] to [43] above, wherein the cell is at least one selected from the group consisting of a neutrophil, an eosinophil, a basophil, a mast cell, a natural killer cell, a natural killer T cell, a cytotoxic T cell and a platelet.
[45] The method described in [44] above, wherein the cell is a mast cell.
[46] A method of controlling the cytokine production, which comprises controlling an intracellular zinc ion concentration in vitro.
[47] The method described in [46] above, wherein the zinc ion concentration is controlled by a zinc ion.
[48] The method described in [47] above, wherein the zinc ion is introduced into a cell through a zinc ionophore.
[49] The method described in [46] above, wherein the zinc ion concentration is controlled by a zinc ion chelator.
[50] The method described in [49] above, wherein the zinc ion chelator is at least one selected from the group consisting of 2,3-dimercapto-1-propanesulfonic acid (DMPS), N,N,N′,N′-tetrakis(2-pyridylmethyl)ethylenediamine (TPEN), ethylenediamine (EDTA), and N-(6-methoxy-8-quinolyl)-p-toluenesulfonamide (TSQ).
[51] The method described in [46] above, wherein the zinc ion concentration is controlled by regulating the expression and/or function of a zinc ion-requiring protein.
[52] The method described in [51] above, wherein the zinc ion-requiring protein is at least one selected from the group consisting of Raf-1, PKCα, GEF-H1, HDAC, SQSTM1, ubiquitin-protein ligase E3, ubiquitin-conjugating enzyme E2, metallothionein, phosphatase, Snail, TRAF6, Paxillin, Zyxin and Jade-1.
[53] The method described in [46] above, wherein the zinc ion concentration is controlled by regulating the expression and/or function of a zinc ion transporter.
[54] The method described in [53] above, wherein the zinc ion transporter is at least one selected from the group consisting of human ZIPs including LIV family and human CDFs.
[55] The method described in any one of [46] to [54] above, wherein the cell is at least one selected from the group consisting of a neutrophil, an eosinophil, a basophil, a mast cell, a natural killer cell, a natural killer T cell, a cytotoxic T cell and a platelet.
[56] The method described in [55] above, wherein the cell is a mast cell.
[57] A method of screening for a compound having a degranulation reaction-controlling action, which comprises measuring an intracellular zinc ion concentration.
[58] The screening method described in [57] above, wherein the cell is at least one selected from the group consisting of a neutrophil, an eosinophil, a basophil, a mast cell, a natural killer cell, a natural killer T cell, a cytotoxic T cell and a platelet.
[59] The screening method described in [58] above, wherein the cell is a mast cell.
[60] A method of screening for a compound having a cytokine production-controlling action, which comprises measuring an intracellular zinc ion concentration.
[61] The screening method described in [60] above, wherein the cell is at least one selected from the group consisting of a neutrophil, an eosinophil, a basophil, a mast cell, a natural killer cell, a natural killer T cell, a cytotoxic T cell and a platelet.
[62] The screening method described in [61] above, wherein the cell is a mast cell.
[63] A method of controlling a degranulation reaction, which comprises administrating an effective amount of a substance capable of controlling an intracellular zinc ion concentration.
[64] A use of a substance capable of controlling an intracellular zinc ion concentration, for the production of an agent for controlling a degranulation reaction.
[65] A method of controlling a cytokine production, which comprises administrating an effective amount of a substance capable of controlling an intracellular zinc ion concentration.
[66] A use of a substance capable of controlling an intracellular zinc ion concentration, for the production of an agent for controlling a cytokine production.
[67] A method for the prophylaxis and/or treatment of an allergic disease and/or an inflammatory disease including an autoimmune disease, which comprises administrating an effective amount of a substance capable of controlling an intracellular zinc ion concentration.
[68] A use of a substance capable of controlling an intracellular zinc ion concentration, for the production of a prophylactic and/or therapeutic drug for an allergic disease and/or an inflammatory disease including an autoimmune disease.

### BRIEF DESCRIPTION OF THE DRAWINGS

FIG. 1 shows the effect of a zinc ion chelator (TPEN) on the antigen stimulation-dependent release (degranulation) of a chemical mediator (β-hexosaminidase) from mast cells.

FIG. 2 shows that the effect of a zinc ion chelator (TPEN) on the antigen stimulation-dependent release (degranulation) of a chemical mediator (β-hexosaminidase) from mast cells can be restored by enforced introduction of zinc ion.

FIG. 3 shows the effect of a zinc ion chelator (TPEN) on the cytokine production of mast cells. The upper shows the effect of TPEN on the production of IL-6, and the lower shows the effect on the production of TNF-α.

FIG. 4 shows the effect of a zinc ion chelator (TPEN) on the type I allergy (passive cutaneous anaphylaxis) response.

### BEST MODE FOR CARRYING OUT THE INVENTION

In the present invention, by “controlling a zinc ion concentration” is not only literally meant making the intracellular zinc ion concentration (amount) increase or decrease, but also meant an effect which can ultimately induce the same phenomena as those caused by an increase or decrease of the intracellular zinc ion concentration, and in such a case, the wording is not limited by the level of the intracellular zinc ion concentration. In the present invention, a cell to be a target of control of the zinc ion concentration is a cell that can release inflammatory chemical mediators such as histamine and/or a cell with an ability to produce cytokines, and exemplified by a neutrophil, an eosinophil, a basophil, a mast cell, a natural killer cell, a natural killer T cell, a cytotoxic T cell, a platelet and the like. Preferably, the cell is a mast cell. Herein, a cell to be a target of such control is sometimes referred to as a “target cell” for convenience.

In the present invention, by “cytokine” is generally meant a molecule which may induce various allergic diseases and various inflammatory diseases including autoimmune diseases, when produced or released in excess, and cytokines include interleukin (IL)-1, IL-2, IL-3, IL-4, IL-5, IL-6, IL-10, IL-12, IL-13, granulocyte colony stimulating factor (G-CSF), granulocyte macrophage colony stimulating factor (GM-CSF), tumor necrosis factor-α (TNF-α), transforming growth factor-β (TGF-β), interferon-γ (IFN-γ), macrophage colony stimulating factor (M-CSF), monocyte chemoattractant protein (MCP-1), MIPIβ, MIPIα, leukemia inhibitory factor (LIF) and the like.

A “substance capable of controlling an intracellular zinc ion concentration” includes a “zinc ion”, a “zinc ion chelator”, a “zinc ionophore”, a “substance regulating the expression and/or function of a zinc ion-requiring protein”, a “substance regulating the expression and/or function of a zinc ion transporter” and the like. The “zinc ion” and “zinc ionophore” can directly increase the intracellular zinc ion concentration of the target cell by addition thereof, whereas the “zinc ion chelator” can directly decrease the zinc ion concentration in the target cell by addition thereof. The “substance regulating the expression and/or function of a zinc ion-requiring protein” and the “substance regulating the expression and/or function of a zinc ion transporter” can induce the same phenomena in the degranulation reaction and/or cytokine production of the target cell as those that are brought about by an increase or decrease of the intracellular zinc ion concentration, by regulating the expression and/or function of a zinc ion-requiring protein or by regulating the expression and/or function of a zinc ion transporter. Namely, they can indirectly control the effect of the zinc ion. When the “substance regulating the expression and/or function of a zinc ion-requiring protein” or the “substance regulating the expression and/or function of a zinc ion transporter” is a substance regulating in the direction of promoting the expression or function, the responsiveness of the cell to zinc ions can be more enhanced, and when the substance is a substance regulating in the direction of suppressing the expression or function, the responsiveness of the cell to zinc ions can be suppressed more. Namely, in the present invention, by “controlling” is meant both positive and negative regulations.

The “zinc ion” is introduced into the target cell through a zinc ionophore. Namely, the zinc ion is introduced in the form of a complex with the zinc ionophore into the target cell. The zinc ionophore includes a variety of compounds commonly used in the art, preferably those commercially available. It is exemplified by zinc pyrithione, heterocyclic amine, dithiocarbamate, vitamins and the like.

The “zinc ion chelator” is a substance that can form a complex with the zinc ion in the target cell, thereby removing the zinc ion from the cell, and includes, for example, 2,3-dimercapto-1-propanesulfonic acid (DMPS), N,N,N′,N′-tetrakis(2-pyridylmethyl)ethylenediamine (TPEN), ethylenediamine (EDTA), N-(6-methoxy-8-quinolyl)-p-toluenesulfonamide (TSQ) and the like. All of them are commercially available. The amount of the zinc ion chelator as a substance capable of controlling a concentration of the zinc ion to be comprised as an active ingredient in the agent for controlling the degranulation reaction and the agent for controlling the cytokine production of the present invention, and the prophylactic and/or therapeutic drug of the present invention for allergic diseases and/or inflammatory diseases including autoimmune diseases (hereinafter, sometimes simply referred to as “prophylactic and/or therapeutic drug”) is appropriately determined within a range in which the control of the intracellular zinc ion concentration is possible, in each agent or drug. One or more types of zinc ion chelators may be comprised therein. When two or more types of zinc ion chelators are comprised, their amounts are appropriately set depending on their types.

The “substance regulating the expression and/or function of a zinc ion-requiring protein” is not particularly limited by its mechanism of action, as long as it can eventually regulate the expression and/or function of a zinc ion-requiring protein, and the regulation may be at the gene level or protein level. The regulation at the gene level includes transcriptional regulation, gene expression regulation and the like. The regulation at the protein level includes metabolic regulation, phosphorylation, dephosphorylation, glycosylation, lipidation, coordinate bond with zinc, degradation, ubiquitination, acetylation and the like. The “zinc ion-requiring protein” is a protein that requires zinc for its functional expression in the degranulation reaction and/or cytokine production in the target cell, and includes, for example, a protein having zinc finger, a protein having RING finger, a protein having LIM domain, a protein having PHD zinc finger and the like. The zinc finger is a motif having a unique nucleic acid-binding ability, which can form a higher-order structure only by coordination of amino acids such as cysteine and histidine to the zinc. For example, a protein having zinc finger is exemplified by a transcription factor Snail which is a master regulator in a phenomenon called epithelial-mesenchymal transition (phenomenon in which cells usually tightly adhering to each other acquire mobility by releasing the adhesion to their adjacent cells and migrate to other sites during morphogenesis in the early development of humans and other organisms and during wound healing and cancer metastasis and the like), PKCA, GEF-H1, HDAC, SQSTM1 and the like. The RING finger may be said to be a variation of the zinc finger and it has been suggested that it is involved in a protein-protein interaction. The reported protein having the RING finger includes those having ubiquitin-protein ligase E3 activity, those exhibiting ubiquitin-conjugating enzyme E2 binding, and those exhibiting TRAF6 (a signaling molecule of TNFα) binding. The LIM domain consists of 60 amino acids with the positions of 6 cysteines and 1 histidine being conserved. Although this domain is structurally very similar to the zinc finger, its DNA binding ability has not been known. It also functions as a domain mediating the binding to PKC. A protein having the LIM domain is exemplified by Paxillin, Zyxin and the like, which are involved in cytoskeletal control. The PHD zinc finger is a zinc finger-like domain in nuclear proteins suggested to be involved in chromatin-mediated transcriptional control and is expected to have the DNA binding ability. A protein having PHD zinc finger is exemplified by Jade-1 which is involved in histone acetylation and the like.

The “substance regulating the expression and/or function of a zinc ion-requiring protein” may be a known compound or a novel compound to be developed in the future. It may be a low-molecular-weight compound or a high-molecular-weight compound. In this context, the low-molecular-weight compound is a compound having a molecular weight of less than approximately 3000, which includes, for example, an organic compound and a derivative thereof and an inorganic compound usually available as a pharmaceutical agent, and refers to a compound and a derivative thereof produced by making full use of organic synthesis or the like, a naturally occurring compound and a derivative thereof, small nucleic acid molecules such as promoter, a variety of metals and the like, and desirably an organic compound and a derivative thereof and a nucleic acid molecule available as a pharmaceutical agent. The high-molecular-weight compound is a compound having a molecular weight of not lower than approximately 3000, which includes, for example, protein, polynucleic acid, polysaccharide and combinations thereof, and is desirably a protein. These low-molecular-weight or high-molecular-weight compounds, if they are known, are commercially available or can be obtained by way of steps such as collection, production and purification according to the respective reported documents. These compounds may be naturally occurring or prepared by genetic engineering, or can also be obtained by semi-synthesis and the like. Specifically, the expression of a zinc ion-requiring protein Snail is regulated by MAPK through TGF-β or FGF (Peinado, H., Quintanilla, M. & Cano, A. Transforming growth factor beta-1 induces snail transcription factor in epithelial cell lines: mechanisms for epithelial mesenchymal transitions. J. Biol. Chem. 278, 21113-23 (2003)). Moreover, Snail is activated by LIV1 (as described below), a zinc ion transporter (Yamashita, S., Miyagi, C., Fukada, T., Kagara, N., Che, Y.-S. & Hirano, T. Zinc transporter LIVI controls epithelial-mesenchymal transition in zebrafish gastrula organizer. Nature 429, 298-302 (2004)). Details for Raf1, GEF-H1, PKCα, phosphatase and metallothionein exemplified as other zinc ion-requiring proteins are respectively described in Jirakulaporn T, Muslin A J. Cation diffusion facilitator proteins modulate Raf-1 activity. J. Biol. Chem. 279, 27807-15 (2004), Krendel M, Zenke F T, Bokoch G M. Nucleotide exchange factor GEF-H1 mediates cross-talk between microtubules and the actin cytoskeleton. Nature Cell Biology 4, 294-301 (2002), Korichneva I, Hoyos B, Chua R, Levi E, Hammerling U. Zinc release from protein kinase C as the common event during activation by lipid second messenger or reactive oxygen. J. Biol. Chem. 277, 44327-31 (2002), Haase H, Maret W. Intracellular zinc fluctuations modulate protein tyrosine phosphatase activity in insulin/insulin-like growth factor-1 signaling. Exp. Cell Research 291, 289-298 (2003), and Seikagaku Jiten (3^{rd} ed.), Tokyo Kagaku Dozin Co., Ltd., pp. 1393 (1998). Details for TRAF6, Paxillin, Zyxin and Jade-1 are respectively described in Kobayashi N, Kadono Y, Naito A, Matsumoto K, Yamamoto T, Tanaka S, Inoue J. Segregation of TRAF6-mediated signaling pathways clarifies its role in osteoclastogenesis. The EMBO J. 20, 1271-1280 (2001), Brown M C, Perrotta J A, Turner C E. Identification of LIM3 as the principal determinant of paxillin focal adhesion localization and characterization of a novel motif on paxillin directing vinculin and focal adhesion kinase binding. J. Cell Biol. 135, 1109-1123 (1996), Sadler I, Crawford A W, Michelsen J W, Beckerle M C. Zyxin and cCRP: two interactive LIM domain proteins associated with the cytoskeleton. J. Cell Biol. 119, 1573-1587 (1992), and Panchenko M V, Zhou M I, Cohen H T. von Hippel-Lindau partner Jade-1 is a transcriptional co-activator associated with histone acetyltransferase activity. J. Biol. Chem. 279, 56032-56041 (2004). Furthermore, details for SQSTM1 and HDAC are respectively described in Joung I, Strominger J L, Shin J. Molecular cloning of a phosphotyrosine-independent ligand of the p56lck SH2 domain. Proc Natl Acad Sci USA, 93, 5991-5995 (1996) and Kawaguchi Y, Kovacs J J, McLaurin A, Vance J M, Ito A, Yao T P. The deacetylase HDAC6 regulates aggresome formation and cell viability in response to misfolded protein stress. Cell 115, 727-738 (2003).

The “substance regulating the expression and/or function of a zinc ion transporter” is not particularly limited by its mechanism of action, as long as it can eventually regulate the expression and/or function of a zinc ion transporter, and the regulation may be at the gene level or protein level. The regulation at the gene level includes transcriptional regulation, gene expression regulation and the like. The regulation at the protein level includes metabolic regulation, phosphorylation, dephosphorylation, glycosylation, lipidation, coordinate bond with zinc, degradation, ubiquitination, acetylation and the like. The “zinc ion transporter” includes, for example, human ZIPs including LIV family (e.g., BAB70848, hZip4, BIGM103, KIAA0062, KIAA1265, hLiv-1, AAH08853, hKE4, XP_{—}208649, hZIP1, hZIP2, hZIP3, BAA92100, BAC04504), human CDFs (e.g., hZnt-5, hZnt-7, hZnt-1, hZnt-6, hZnt-3, hZnt-2, hZnt-8, hZnt-4, hZnt-3, hZnt-9) and the like. LIV1 was originally identified as an estrogen-controlled breast cancer protein and recently has been revealed to belong to a ZIP zinc ion transporter subfamily (Zrt⁻, Irt-like protein) called LZT (LIV-1 subfamily of ZIP zinc ion transporter) (Taylor, K. M. & Nicholson, R. I. The LZT proteins; the LIV-1 subfamily of zinc transporters. Biochim. Biophys. Acta 1611, 16-30 (2003)), and to function as a zinc ion transporter (Taylor, K. M., Morgan, H. E., Johnson, A., Hadley, L. J. & Nicholson, R. I. Structure-function analysis of LIV-1, the breast cancer-associated protein that belongs to a new subfamily of zinc transporters. Biochem. J. 375, 51-9 (2003)). In addition, CDF (cation diffusion facilitator) and the like are included.

For example, the expression of the zinc ion transporter LIV1 is regulated by STAT3 (Yamashita, S., Miyagi, C., Fukada, T., Kagara, N., Che, Y.-S. & Hirano, T. Zinc transporter LIVI controls epithelial-mesenchymal transition in zebrafish gastrula organizer. Nature 429, 298-302 (2004)).

The “substance regulating the expression and/or function of a zinc ion transporter” may be a known compound or a novel compound to be developed in the future. It may be a low-molecular-weight compound or a high-molecular-weight compound. In this context, the low-molecular-weight compound is a compound having a molecular weight of less than approximately 3000, which includes, for example, an organic compound and a derivative thereof and an inorganic compound usually available as a pharmaceutical agent, and refers to a compound and a derivative thereof produced by making full use of organic synthesis or the like, a naturally occurring compound and a derivative thereof, small nucleic acid molecules such as promoter, a variety of metals and the like, and desirably an organic compound and a derivative thereof and a nucleic acid molecule available as a pharmaceutical agent. The high-molecular-weight compound is a compound having a molecular weight of not lower than approximately 3000, which includes, for example, protein, polynucleic acid, polysaccharide and combinations thereof, and is desirably a protein. These low-molecular-weight or high-molecular-weight compounds, if they are known, are commercially available or can be obtained by way of steps such as collection, production and purification according to the respective reported documents. These compounds may be naturally occurring or prepared by genetic engineering, or can also be obtained by semi-synthesis and the like.

Since the zinc ion transporter LIV1 enhances the activity (particularly, repressor activity) of the zinc ion-requiring protein Snail (Yamashita, S., Miyagi, C., Fukada, T., Kagara, N., Che, Y.-S. & Hirano, T. Zinc transporter LIVI controls epithelial-mesenchymal transition in zebrafish gastrula organizer. Nature 429, 298-302 (2004)), an example of the “substance regulating the expression and/or function of a zinc ion-requiring protein” in the present invention includes the following:

(1) DNA according to any of the following a) to d):

a) DNA encoding a protein (LIV1 protein) having an amino acid sequence described in SEQ ID NO: 2, 4, or 6;

b) DNA (LIV1 gene) consisting of a sequence described in SEQ ID NO: 1, 3, or 5;

c) DNA encoding a protein (protein analogous to LIV1) having an amino acid sequence described in SEQ ID NO: 2, 4, or 6 with substitution, deletion, insertion and/or addition of one or more amino acid sequences therein;

d) DNA hybridizing to a sequence described in SEQ ID NO: 1, 3, or 5 under stringent condition;

(2) a vector in which DNA according to any of the following a) to d) is inserted:

a) DNA encoding a protein having an amino acid sequence described in SEQ ID NO: 2, 4, or 6;

b) DNA consisting of a sequence described in SEQ ID NO: 1, 3, or 5;

c) DNA encoding a protein having an amino acid sequence described in SEQ ID NO: 2, 4, or 6 with substitution, deletion, insertion and/or addition of one or more amino acid sequences therein;

d) DNA hybridizing to a sequence described in SEQ ID NO: 1, 3, or 5 under stringent condition;

(3) a protein encoded by DNA according to any of the following a) to d):

a) DNA encoding a protein having an amino acid sequence described in SEQ ID NO: 2, 4, or 6;

b) DNA consisting of a sequence described in SEQ ID NO: 1, 3, or 5;

c) DNA encoding a protein having an amino acid sequence described in SEQ ID NO: 2, 4, or 6 with substitution, deletion, insertion and/or addition of one or more amino acid sequences therein;

d) DNA hybridizing to a sequence described in SEQ ID NO: 1, 3, or 5 under stringent condition;

(4) An antisense oligonucleotide whose target sequence is DNA consisting of a sequence described in SEQ ID NO: 1, 3, or 5;

(5) double-stranded RNA having a sequence identical or similar to a portion of DNA consisting of a sequence described in SEQ ID NO: 1, 3, or 5;

(6) an antibody against a protein having an amino acid sequence described in SEQ ID NO: 2, 4, or 6;

(7) a substance (including natural and non-natural products) that associates with a protein having an amino acid sequence described in SEQ ID NO: 2, 4, or 6 and regulates a function of the protein.

The substances (1) to (3) positively regulate the expression and/or function of the zinc ion-requiring protein Snail (enhancing the expression and/or activating the function), and the substances (4) and (5) negatively regulate the expression and/or function of Snail (reducing the expression and/or suppressing the function).

The LIV1 protein can be prepared by a variety of methods well known to those skilled in the art. For example, the protein can be produced by a transformant and obtained by purification thereof, wherein the transformant bears a vector in which DNA (LIV1 gene) consisting of a base sequence described in SEQ ID NO: 1, 3, or 5 is inserted. The vector to be used can be appropriately selected depending on translation systems used for the protein production. Moreover, LIV1 is known to be expressed in hormonal tissues such as the breast, prostate, pituitary gland and brain (Taylor, K. M. & Nicholson, R. I. The LZT proteins; the LIV-1 subfamily of zinc transporters. Biochim. Biophys. Acta 1611, 16-30 (2003)). The LIV1 protein can be purified from LIV1-expressing cell extracts by preparing anti-LIV1 protein antibodies by a well known method and producing an affinity column with the antibodies.

A protein analogous to LIV1 is a protein capable of regulating the activity of a zinc ion-requiring protein (e.g., Snail) and can include, for example, a protein consisting of an amino acid sequence described in SEQ ID NO: 2, 4, or 6 with substitution, deletion, insertion and/or addition of one or more amino acid sequences therein, and a protein encoded by DNA hybridizing to DNA consisting of a base sequence described in SEQ ID NO: 1, 3, or 5 under stringent condition.

The protein analogous to LIV1 can be prepared for example, by a method involving performing hybridization utilizing a base sequence encoding LIV1, producing a transformant with the obtained DNA with high homology and allowing the transformant to produce the desired protein. An example of a method for obtaining the DNA with high homology can include a method involving performing hybridization under stringent hybridization condition using a portion of the base sequence described in SEQ ID NO: 1, 3, or 5 as a probe to obtain such a DNA from cells or the like of human or non-human vertebrate.

Those skilled in the art can appropriately select the above stringent hybridization condition. By way of example, prehybridization is performed overnight at 42° C. in a hybridization solution containing 25% formamide or 50% formamide for more stringent condition, 4×SSC, 50 mM Hepes pH 7.0, 10×Denhardt's solution, and 20 μg/mL denatured salmon sperm DNA, and then hybridization is performed by adding a labeled probe and incubating overnight at 42° C. Subsequent washing can be performed under condition of a washing liquid and temperature of approximately “1×SSC, 0.1% SDS, at 37° C.”, approximately “0.5×SSC, 0.1% SDS, at 42° C.” for more stringent condition, and approximately “0.2×SSC, 0.1% SDS, at 65° C.” for even more stringent condition. Thus, it can be expected that the more stringent the washing condition of the hybridization is, the higher the homology of the isolated DNA to the probe sequence is. However, the combinations of the SSC, SDS and temperature conditions are described for purposes of only exemplification, and those skilled in the art can appropriately combine the above-described or other determinants of the stringency of hybridization (e.g., probe concentration, probe length, hybridization reaction period and the like), thereby achieving a stringency similar to those described above.

A polypeptide encoded by the DNA isolated using such a hybridization technique generally has high homology to LIV1 in the amino acid sequence. The high homology refers to at least 40% or higher, preferably 60% or higher, more preferably 80% or higher, even more preferably 90% or higher, even still more preferably 95% or higher, particularly preferably 97% or higher (e.g., 98 to 99%) of sequence homology. An amino acid sequence identity can be determined by, for example, algorithm BLAST (Karlin and Altschul, Proc. Natl. Acad. Sci. USA 87: 2264-2268, 1990; and Proc. Natl. Acad. Sci. USA 90: 5873-5877, 1993). Based on this algorithm, a program called BLASTX has been developed (Altschul et al., J. Mol. Biol. 215: 403-410, 1990). When amino acid sequences are analyzed with the use of BLASTX, parameters are set to, for example, score=50 and word length=3. When BLAST and Gapped BLAST programs are used, the respective default parameters of the programs are used. The specific ways of these analysis methods are known (http://www.ncbi.nlm.nih.gov.)

The protein analogous to LIV1 may also be prepared by another known means. For example, LIV1 DNA is artificially modified by deletion mutant production using exonuclease and site-directed mutagenesis such as cassette mutagenesis, and the modified LIV1 DNA can be used to prepare the desired protein.

Another preferable example of the substance available as the “substance regulating the expression and/or function of a zinc ion-requiring protein” can include DNA consisting of the sequence described in SEQ ID NO: 1, 3, or 5. The above described DNA encodes LIV1 protein, and when introduced into cells, serves as a substance capable of indirectly regulating the activity of a zinc ion-requiring protein such as Snail, through LIV1 protein expression.

The above described DNA can also be prepared from a cDNA of a LIV1-expressing cell by hybridization techniques well known to those skilled in the art using a portion of the sequence described in SEQ ID NO: 1, 3, or 5 as a probe. The DNA can also be obtained from mRNA by RT-PCR using a portion of the sequence described in SEQ ID NO: 1, 3, or 5 as a primer. Alternatively, the DNA may also be synthesized artificially with a commercially available DNA synthesizer.

Furthermore, DNA similar to the above DNA is also an example of the “substance regulating the expression and/or function of a zinc ion-requiring protein”. The similar DNA can include DNA hybridizing to the sequence described in SEQ ID NO: 1, 3, or 5 under stringent condition and encoding the substance regulating the expression and/or function of a zinc ion-requiring protein, for example, a protein capable of regulating the activity of Snail. The DNA can be prepared as previously described above.

When each DNA described above is used, the DNA can be inserted into an appropriate vector and used. The DNA inserted into the vector is also one of the aspects of the present invention. As a vector to be used, a suitable vector can be selected depending on the purpose. Particularly, the vector can include mammal-derived vectors (e.g., pcDNA3 (manufactured by Invitrogen), pEGF-BOS (Nucleic Acids. Res., 18(17), p. 5322, 1990), pEF, pCDM8, pCXN), insect cell-derived vectors (e.g., “Bac-to-BAC baculovirus expression system” (manufactured by Invitrogen), pBacPAK8), plant-derived expression vectors (e.g., pMH1, pMH2), animal virus-derived vectors (e.g., pHSV, pMV, pAdexLcw), retrovirus-derived vectors (e.g., pZIPneo), yeast-derived vectors (e.g., “Pichia Expression Kit” (manufactured by Invitrogen), pNV11, SP-Q01), Bacillus subtilis-derived vectors (e.g., pPL608, pKTH50), Escherichia coli vectors (M13-type vectors, pUC-type vectors, pBR322, pBluescript, pCR-Script) and the like. In the present invention, it is preferred to use a vector capable of being expressed in a mammal cell and to use an expression vector. The vector can be introduced into a cell by a method selected from, for example, calcium phosphate method (Virology, Vol. 52, p. 456, 1973), DEAE dextran method, a method using cationic liposome DOTAP (manufactured by Roche Diagnostics), electroporation method (Nucleic Acids Res., Vol. 15, p. 1311, 1987), lipofection method (J. Clin. Biochem. Nutr., Vol. 7, p. 175, 1989), introduction method by viral infection (e.g., pMX, pMSCV; Sci. Am., p. 34, 1994), a particle gun and the like.

One example of the “substance regulating the expression and/or function of a zinc ion-requiring protein”, for example, an agent suppressing the activity of Snail can include an antisense oligonucleotide targeting DNA or mRNA encoding LIV1. It is believed that the antisense oligonucleotide against the DNA encoding LIV1 prevents the expression of an endogenous LIV1 gene and negatively control the Snail activity. Such an antisense oligonucleotide can include an antisense oligonucleotide whose target sequence is DNA consisting of a sequence described in SEQ ID NO: 1, 3, or 5 or mRNA sequence generated from the DNA sequence. An example thereof can include an oligonucleotide described in SEQ ID NO: 7 or 8. In addition, any antisense oligonucleotide that can hybridize to any site in the DNA consisting of the above sequence described in SEQ ID NO: 1, 3, or 5, and the like, and can effectively inhibit LIV1 expression is included in the antisense oligonucleotide of the present invention. Such an antisense oligonucleotide is not necessarily completely complementary to the DNA consisting of the above sequence described in SEQ ID NO: 1, 3, or 5, or the corresponding mRNA.

The above described antisense oligonucleotide can be inserted into a suitable vector depending on the purposes and used. For example, for the purpose of applying it to a gene therapy, the vector may be selected appropriately from viral vectors such as retroviral, adenoviral and vaccinia viral vectors, and non-viral vectors such as cationic liposomes and ligand-DNA complexes, and the like. It can also be administered as a naked plasmid DNA (naked pDNA) together with a large volume of aqueous solutions without the use of carriers.

Another example of the “substance regulating the expression and/or function of a zinc ion-requiring protein”, particularly the agent suppressing the activity of Snail can include double-stranded RNA having a sequence identical or similar to a portion of DNA encoding LIV1. A double-stranded RNA having a sequence identical or similar to a target gene sequence is capable of inducing RNA interference (RNAi) that inhibits the expression of the target gene. The RNAi refers to a phenomenon in which when double-stranded RNA (dsRNA) is introduced into a cell, an intracellular mRNA corresponding to the RNA sequence is specifically degraded and as a result is not expressed as a protein. The double-stranded RNA may be entirely composed of a region forming double strand or may have a partial region forming single strand (e.g., at both ends or either end) and the like. Thus, the double-stranded RNA of the present invention may also contain a region that is not double-stranded. An oligo RNA used in RNAi is often 10- to 100-bp RNA, and usually 19- to 23-bp RNA. The RNAi can be performed according to the descriptions of Nature, Vol. 391, p. 806, 1998, Proc. Natl. Acad. Sci. USA Vol. 95, p. 15502, 1998, Nature, Vol. 395, p. 854, 1998, Proc. Natl. Acad. Sci. USA Vol. 96, p. 5049, 1999, Cell, Vol. 95, p. 1017, 1998, Proc. Natl. Acad. Sci. USA Vol. 96, p. 1451, 1999, Proc. Natl. Acad. Sci. USA Vol. 95, p. 13959, 1998, Nature Cell Biol., Vol. 2, p. 70, 2000, and the like.

The “substance regulating the expression and/or function of a zinc ion transporter” is, for example, when the substance is LIV1, exemplified by those similar to the substances above described for the “substance regulating the expression and/or function of a zinc ion-requiring protein”.

When the “zinc ion transporter” is other than LIV1, for example, hZnt-1, hZnt-2, hZnt-5, hZnt-6, hZnt-7, hZIP1, hZIP2, hZIP3, BAA92100, BAC04504, hLiv-1, hKE4, KIAA1265, KIAA0062, or hZip4, a variety of “substances regulating the expression and/or function of a zinc ion transporter” or “substances regulating the expression and/or function of a zinc ion-requiring protein” can be prepared in the same way as for LIV1, based on the known amino acid sequence or base sequence of the zinc ion transporter.

Information about the respective amino acid sequences and base sequences can be obtained from a variety of databases browsable in NCBI and the like.

The agent for controlling the degranulation reaction, the agent for controlling the cytokine production, as well as the prophylactic and/or therapeutic drug for allergic diseases and/or inflammatory diseases including autoimmune diseases according to the present invention comprise, if desired, pharmaceutically acceptable excipients and additives, in addition to the above described series of active ingredients (zinc ion, zinc ionophore, zinc ion chelator, substance regulating the expression and/or function of a zinc ion-requiring protein, and substance regulating the expression and/or function of a zinc ion transporter). The pharmaceutically acceptable excipients and additives include carriers, binders, flavors, buffers, thickeners, coloring agents, stabilizers, emulsifiers, dispersants, suspending agents, preservatives and the like. Moreover, different types of “substances controlling the zinc ion concentration” can be used in combination as active ingredients.

The pharmaceutically acceptable carriers include, for example, magnesium carbonate, magnesium stearate, talc, sugar, lactose, pectin, dextrin, starch, gelatin, tragacanth, methylcellulose, sodium carboxymethylcellulose, low-melting wax, cocoa butter and the like.

Furthermore, tablets can be made, if necessary, into tablets coated with usual coatings, for example sugar-coated tablets, enteric-coated tablets, film-coated tablets, or two-layer tablets or multi-layer tablets. Powders can be prepared with pharmaceutically acceptable bases for powders. The bases include talc, lactose, starch and the like. Drops can be prepared together with aqueous or non-aqueous bases and one or more pharmaceutically acceptable dispersants, suspending agents, solubilizers, and the like. Capsules can be prepared by filling with a compound serving as an active ingredient together with a pharmaceutically acceptable carrier. The compound can be mixed with or without a pharmaceutically acceptable excipient and filled into capsules. Cachets can also be prepared in a similar way. When the present invention is prepared as a suppository, the suppository is prepared together with bases such as vegetable oil (e.g., castor oil, olive oil, peanut oil), mineral oil (e.g., petrolatum, white petrolatum), waxes, and partially or fully synthesized glycerin fatty acid ester, by an approach generally used.

Liquid formulations for injection include solutions, suspensions, emulsions and the like. Examples thereof include aqueous solutions, water-propylene glycol solutions and the like. The liquid formulations can also be produced in the form of a solution of polyethylene glycol and/or propylene glycol, which may contain water.

Liquid formulations suitable for oral administration can be produced by adding the compound serving as an active ingredient and, if necessary, a coloring agent, flavor, stabilizer, sweetening agent, solubilizer, thickener, or the like, to water. Alternatively, the liquid formulations suitable for oral administration can be produced by adding the compound and a dispersant to water to give a viscous liquid. Examples of the thickener include pharmaceutically acceptable natural or synthetic gum, resin, methylcellulose, sodium carboxymethylcellulose, known suspending agents and the like.

Agents for local administration include the liquid formation described above, and cream, aerosol, spray, powder, lotion, ointment and the like. The above described agents for local administration can be produced by mixing a compound serving as an active ingredient with pharmaceutically acceptable diluents and carriers. The ointment and cream can be prepared, for example, by adding thickeners and/or gelling agents to an aqueous or oily base. Examples of the base include water, liquid paraffin, vegetable oil and the like. Examples of the thickener include soft paraffin, aluminum stearate, cetostearyl alcohol, propylene glycol, polyethylene glycol, lanolin, hydrogenated lanolin, beeswax and the like. To the agent for local administration can be added, if necessary, a preservative such as methyl hydroxybenzoate, propyl hydroxybenzoate, chlorocresol, benzalkonium chloride and the like, or a bacterial growth inhibitor. The lotion can be prepared by adding one or more pharmaceutically acceptable stabilizers, suspending agents, emulsifiers, dispersants, thickeners, coloring agents, flavors, and the like, to an aqueous or oily base.

The thus-obtained agent for controlling the degranulation reaction, agent for controlling the cytokine production, as well as prophylactic and/or therapeutic drug for allergic disease and/or inflammatory disease including autoimmune disease of the present invention are administered orally or parenterally.

When administered orally, they can be administered in a dosage form commonly used in the art. When administered parenterally, they can be administered in a dosage form such as agent for local administration (e.g., transdermal agent), agent for rectal administration, injectable, transnasal agent and the like.

Examples of the oral agent or agent for rectal administration include capsule, tablet, pill, powder, drop, cachet, suppository, liquid formulation and the like. Examples of the injectable include aseptic solutions or suspensions and the like. Examples of the agent for local administration include cream, ointment, lotion, transdermal agent (general patch agent or matrix agent) and the like.

The dosage and the number of administration may vary depending on the type of the substance capable of controlling the zinc ion concentration used, the condition, age and body weight of a patient, a dosage form and the like, and can be determined appropriately.

All of the “zinc ion”, “zinc ion chelator”, “substance regulating the expression and/or function of a zinc ion-requiring protein”, and “substance regulating the expression and/or function of a zinc ion transporter” can provide the control of the zinc ion concentration in cells, particularly cells capable of degranulating chemical mediators and/or cells with an ability to produce cytokines, and provide an effect equivalent thereto to the cells in mammals including human as well as cattle, horse, dog, mouse, rat and the like. Therefore, they can prevent and/or treat a variety of allergic diseases and inflammatory diseases including autoimmune diseases in which the cells capable of degranulating chemical mediators and/or cells with an ability to produce cytokines are involved. When they have a suppressive action on the degranulation, they will suppress the expression of symptoms such as increased vascular permeability, smooth muscle contraction, increased glandular secretion, vasodilation and the like due to type I allergic reaction and improve the symptoms, by an antihistaminic action and the like as a result of the above mentioned action. A compound having a cytokine production-controlling action can repair abnormalities in the cells with an ability to produce cytokines, for example the dysfunction of Th1 cells and Th2 cells, subpopulations of helper T cells. It is believed that the dysfunction of the Th1 cells causes autoimmune diseases, while the dysfunction of the Th2 cells causes allergic diseases. Moreover, such compound can also control the cytokine production and release in mast cells and are effective for a variety of allergic diseases and inflammatory diseases including autoimmune diseases. Thus, the present invention can be used to prevent and treat each disease associated with type I allergic reaction and/or each disease based on the imbalance between Th1 and Th2, for example, anaphylactic shock, allergic rhinitis, allergic conjunctivitis, bronchial asthma, urticaria, atopic dermatitis and the like. In addition, the present invention is quite useful in the prophylaxis and treatment of allergic diseases and/or inflammatory diseases including autoimmune diseases, for example, allergic rhinitis and allergic inflammation caused by cedar pollen, house dust, molds, mites, or hair, skin or feces of pets, and systemic lupus erythematosus, mixed connective tissue disease, rheumatoid arthritis, Sjogren's syndrome, rheumatic fever, Goodpasture syndrome, Basedow disease, Hashimoto disease, Addison disease, autoimmune hemolytic anemia, idiopathic thrombocytopenic purpura, myasthenia gravis, ulcerative colitis, Crohn disease, sympathetic ophthalmia, multiple sclerosis, psoriasis, hepatitis and the like.

Based on the findings obtained by the present inventors that the degranulation reaction and/or cytokine production can be controlled by controlling the zinc ion concentration, it is possible to screen for a compound having a degranulation reaction-controlling action and/or cytokine production-controlling action by measuring an intracellular zinc ion concentration. This screening method is performed by, for example, the following steps:

1) dividing cells, in which the control of the degranulation and/or control of cytokine production are desired, into two groups, and treating one group with a test compound (the remaining untreated group is used as a control);

2) measuring the respective intracellular zinc ion concentrations for the treated cells and the untreated control cells;

3) selecting a test compound that has significantly altered the intracellular zinc ion concentrations of the treated cells as compared with the intracellular zinc ion concentrations of the control cells, and determining the test compound as a compound having a degranulation reaction-controlling action and/or a cytokine production-controlling action.

The cells in which the control of the degranulation and/or control of cytokine production is desired are cells whose degranulation and/or cytokine production cause allergic disease and inflammatory diseases including autoimmune diseases, and include, for example, neutrophil, eosinophil, basophil, mast cell, natural killer cell, natural killer T cell, cytotoxic T cell, platelet and the like. The mast cell is preferable. The cells are randomly divided into two groups, one of which is treated with a test compound. The other is untreated and used as a control cell. Furthermore, a compound known to influence the zinc ion concentration, for example, the zinc ion chelator TPEN and the like, may be used as a positive control compound. The test compound may be a known compound or a novel compound to be developed in the future. It may be a low-molecular-weight compound or a high-molecular-weight compound. In this context, the low-molecular-weight compound is a compound having a molecular weight of less than approximately 3000, which includes, for example, an organic compound and a derivative thereof and an inorganic compound usually available as a pharmaceutical agent, and refers to a compound and a derivative thereof produced by making full use of organic synthesis or the like, a naturally occurring compound and a derivative thereof, small nucleic acid molecules such as promoter, a variety of metals and the like, and desirably an organic compound and a derivative thereof and a nucleic acid molecule available as a pharmaceutical agent. The high-molecular-weight compound is a compound having a molecular weight of not lower than approximately 3000, which includes, for example, protein, polynucleic acid, polysaccharide and combinations thereof, and is desirably a protein. These low-molecular-weight or high-molecular-weight compounds, if they are known, are commercially available or can be obtained by way of steps such as collection, production and purification according to the respective reported documents. These compounds may be naturally occurring or prepared by genetic engineering, or can also be obtained by semi-synthesis and the like.

The period of time of the treatment of cells with the test compound is appropriately set depending on the types and concentrations of cells and test compounds to be used. When the positive control compound such as TPEN is used, the treatment can be performed using, as a guide, a change in the zinc ion concentration when treated with the control compound. The concentration of the test compound for treating cells is also appropriately set depending on the types of the cells and test compounds to be used, as well as the treatment period.

The measurement of the intracellular zinc ion concentration can be performed utilizing a method routinely practiced in the art or a method pursuant thereto. The concentration can be measured, for example, by direct measurement by atomic absorption method (flame method) or with a spectrophotofluorometer using a specific probe (e.g., fluorescent reagent).

### EXAMPLES

Hereinafter, although the present invention will be described in more detail with reference to Examples, these Examples do not limit the scope of the present invention by any means. All the publications cited throughout the present application are incorporated herein by reference. The reagents, apparatuses and materials used in the present invention are commercially available unless otherwise specified.

### Example 1

N,N,N′,N′-tetrakis(2-pyridylmethyl)ethylenediamine (TPEN; SIGMA, P4413), a zinc ion chelator, was used to investigate the role of zinc ions in antigen stimulation-dependent chemical mediator release from mast cells.

The mast cells used were bone marrow-derived mast cells whose differentiation was induced by culturing bone marrow cells with interleukin-3 for 5 weeks. The obtained bone marrow-derived mast cells (1×10⁷ cells) were sensitized with 1 μg/mL IgE antibody (SPE-7 clone; available from SIGMA) for 6 hours. After sensitization, the cells were resuspended in Tyrode's buffer (pH 7.4). The composition of the Tyrode's buffer is as described in Table 1.

TABLE 1

| | | |
|---|---|---|
| 10 mM HEPES | 1.2 | g |
| 130 mM NaCl | 3.8 | g |
| 5 mM KCl | 0.19 | g |
| 1.4 mM CaCl₂ | 0.1 | g |
| 1 mM MgCl₂ | 0.5 | g |
| 5.6 mM glucose | 0.5 | g |
| 0.1% BSA | | |
| Total | 500 | mL |

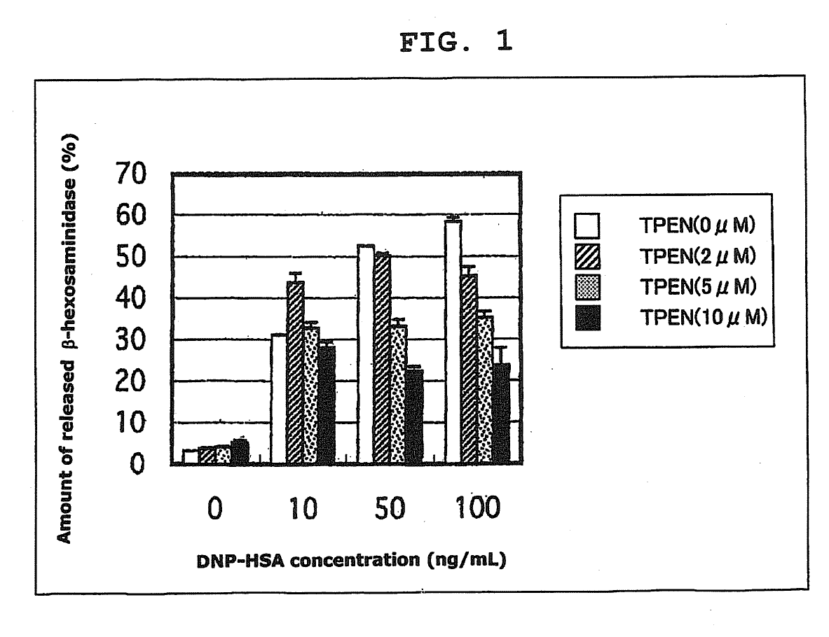

The obtained mast cell suspension was stimulated with an antigen (DNP-HSA; dinitrophenyl conjugated Human Serum Albumin; available from SIGMA) at each concentration (shown in FIG. 1) for 30 minutes. To investigate the effect of zinc ions, a zinc ion chelator TPEN (0, 2, 5 and 10 μM) was allowed to coexist with the cells for 2 hours during the sensitization period and for 30 minutes of the stimulation. Chemical mediator β-hexosaminidase released from the mast cells was measured. The activity of β-hexosaminidase was measured with an absorption spectrometer at wavelength of 405 nm by using 4-nitrophenyl N-acetyl-β-D-glucosaminide (available from SIGMA) as a substrate. The extracellular release of entire β-hexosaminidase present in the cell was defined as 100%.

The results are shown in FIG. 1. The concentration-dependent suppressive effect on the chemical mediator release was achieved by the addition of TPEN.

### Example 2

Whether the suppressive effect of TPEN on the chemical mediator release of mast cells confirmed in Example 1 depends on zinc ions was examined.

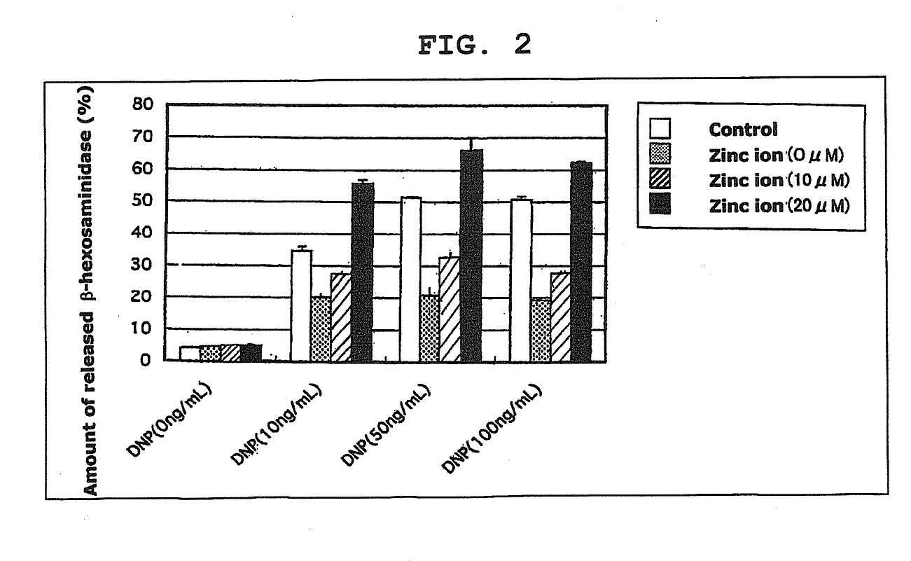

As mast cells sensitized with IgE antibody, the cells prepared in the same way as in Example 1 were used. After sensitization, the mast cells were resuspended in Tyrode's buffer. The mast cell suspension was stimulated with an antigen (DNP-HSA) at each concentration (shown in FIG. 2) for 30 minutes. A zinc ion chelator TPEN (10 μM) was allowed to coexist with the cells for 2 hours during the sensitization period and for 30 minutes of the stimulation. To introduce zinc ions into the cells, the cells were treated with 1 μM pyrithione (Molecular probes, p-24193) during the stimulation, and zinc ions were added at each concentration (shown in FIG. 2) to Tyrode's buffer. The activity of β-hexosaminidase was measured in the same way as in Example 1. TPEN-untreated cells were used as a control, and the extracellular release of entire β-hexosaminidase present in the cell was defined as 100%.

The results are shown in FIG. 2. The suppressive effect of TPEN on the chemical mediator release was restored by the addition of the zinc ions.

### Example 3

The role of zinc ions in the cytokine production of mast cells was examined by using a zinc ion chelator TPEN (SIGMA, P4413).

As mast cells sensitized with IgE antibody, the cells prepared in the same way as described in Example 1 were used. After sensitization, the mast cells were resuspended in Tyrode's buffer. The mast cell suspension was stimulated with an antigen (DNP-HSA) at a concentration of 100 ng/ml for 6 hours. To investigate the effect of zinc ions, a zinc ion chelator TPEN (0, 2, 5 and 10 μM) was allowed to coexist with the cells for 2 hours during sensitization and for 6 hours of the stimulation.

The concentrations of cytokines (IL-6 and TNF-α) in the supernatant were determined using ELISA (BIOSOURCE).

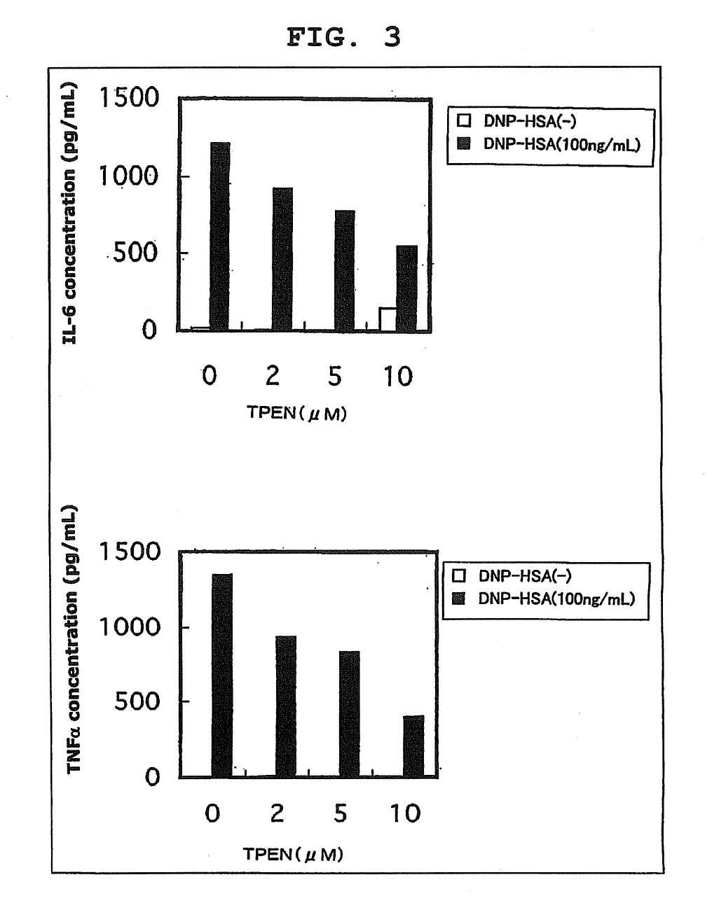

The results are shown in FIG. 3. The dose dependent suppression of the cytokine production was achieved by the addition of TPEN.

### Example 4

Whether a zinc ion chelator exhibits suppressive effect in an allergic disease model mouse was examined.

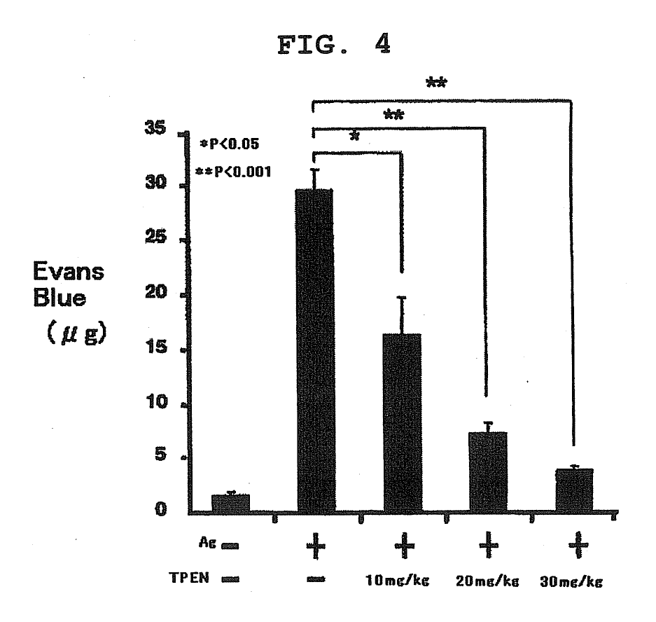

Mouse (6 week-old female, Balb/c Ajc1, CLEA Japan) was sensitized by subcutaneously administering 0.5 μg of IgE antibody (SPE-7, SIGMA) into the ear in order to induce passive cutaneous anaphylaxis. 12 hours later, TPEN (SIGMA, P4413) was intraperitoneally administered to the mouse at each dose as shown in FIG. 4. 30 minutes later, passive cutaneous anaphylaxis was induced by intravenous injection of 250 μg of antigen DNP-BSA (COSMOBIO) and 1.25 mg of Evans blue (SIGMA) into the tail of the mouse. In order to digitalize Evans blue dye leakage, the dye was extracted from the ear of the mouse using formamide. The extracted dye was measured by absorbance at wavelength of 620 nm. The amount of dye in each experiment was calculated from the analytical curve obtained by known amounts of Evans blue.

The results are shown in FIG. 4. The dye leakage of Evans blue was suppressed dose-dependently by the zinc ion chelator. Thus, the results show that the zinc ion chelator suppresses type I allergy (passive cutaneous anaphylaxis) response in vivo.

### Free-Text of Sequence Listing

SEQ ID No: 7: Antisense sequence
SEQ ID No: 8: Antisense sequence

### INDUSTRIAL APPLICABILITY

An agent for controlling a degranulation reaction of the present invention which can control the release of chemical mediators from cells, an agent for controlling a cytokine production of the present invention which can control a cytokine production in cells, a prophylactic and/or therapeutic drug for an allergic disease and/or an inflammatory disease including an autoimmune disease, a method of controlling a degranulation reaction, and a method of controlling a cytokine production, by controlling an intracellular zinc ion concentration by using a zinc ion and a zinc ion chelator, or by regulating the expression and/or function of a zinc ion-requiring protein and a zinc ion transporter, are fundamentally different from conventional anti-allergic agents whose mechanism of action is to suppress the action of the released histamine and the like on target cells. Thus, it is possible to suppress both the various chemical mediators and the cytokines released from mast cell and the like at the same time. Moreover, it is possible to avoid side effects caused by the conventional anti-allergic agents and anti-inflammatory agents such as steroid and the like.

This application is based on the Japanese patent application No. 2005-023196 (filed Jan. 31, 2005), and the contents of which is hereby incorporated entirely by reference.

1. An agent for controlling a degranulation reaction, which comprises a substance capable of controlling an intracellular zinc ion concentration as an active ingredient.

2.-3. (canceled)

4. The agent for controlling the degranulation reaction according to claim 1, wherein the substance capable of controlling the zinc ion concentration is a zinc ion chelator.

5. The agent for controlling the degranulation reaction according to claim 4, wherein the zinc ion chelator is at least one selected from the group consisting of 2,3-dimercapto-1-propanesulfonic acid (DMPS), N,N,N′,N′-tetrakis(2-pyridylmethyl)ethylenediamine (TPEN), ethylenediamine (EDTA), and N-(6-methoxy-8-quinolyl)-p-toluenesulfonamide (TSQ).

6.-9. (canceled)

10. The agent for controlling the degranulation reaction according to claim 1, wherein the cell is at least one selected from the group consisting of a neutrophil, an eosinophil, a basophil, a mast cell, a natural killer cell, a natural killer T cell, a cytotoxic T cell and a platelet.

11. The agent for controlling the degranulation reaction according to claim 10, wherein the cell is a mast cell.

12. An agent for controlling a cytokine production, which comprises a substance capable of controlling an intracellular zinc ion concentration as an active ingredient.

13.-14. (canceled)

15. The agent for controlling the cytokine production according to claim 12, wherein the substance capable of controlling the zinc ion concentration is a zinc ion chelator.

16. The agent for controlling the cytokine production according to claim 15, wherein the zinc ion chelator is at least one selected from the group consisting of 2,3-dimercapto-1-propanesulfonic acid (DMPS), N,N,N′,N′-tetrakis(2-pyridylmethyl)ethylenediamine (TPEN), ethylenediamine (EDTA), and N-(6-methoxy-8-quinolyl)-p-toluenesulfonamide (TSQ).

17.-20. (canceled)

21. The agent for controlling the cytokine production according to claim 12, wherein the cell is at least one selected from the group consisting of a neutrophil, an eosinophil, a basophil, a mast cell, a natural killer cell, a natural killer T cell, a cytotoxic T cell and a platelet.

22. The agent for controlling the cytokine production according to claim 21, wherein the cell is a mast cell.

23. A prophylactic and/or therapeutic drug for an allergic disease and/or an inflammatory disease including an autoimmune disease, which comprises the agent for controlling the degranulation reaction according to claim 1.

24. A prophylactic and/or therapeutic drug for an allergic disease and/or an inflammatory disease including an autoimmune disease, which comprises a substance capable of controlling an intracellular zinc ion concentration as an active ingredient.

25.-26. (canceled)

27. The prophylactic and/or therapeutic drug according to claim 24, wherein the substance capable of controlling the zinc ion concentration is a zinc ion chelator.

28. The prophylactic and/or therapeutic drug according to claim 27, wherein the zinc ion chelator is at least one selected from the group consisting of 2,3-dimercapto-1-propanesulfonic acid (DMPS), N,N,N′,N′-tetrakis(2-pyridylmethyl)ethylenediamine (TPEN), ethylenediamine (EDTA), and N-(6-methoxy-8-quinolyl)-p-toluenesulfonamide (TSQ).

29.-32. (canceled)

33. The prophylactic and/or therapeutic drug according to claim 24, wherein the cell is at least one selected from the group consisting of a neutrophil, an eosinophil, a basophil, a mast cell, a natural killer cell, a natural killer T cell, a cytotoxic T cell and a platelet.

34. The prophylactic and/or therapeutic drug according to claim 33, wherein the cell is a mast cell.

35. A method of controlling the degranulation reaction, which comprises controlling an intracellular zinc ion concentration in vitro.

36.-45. (canceled)

46. A method of controlling the cytokine production, which comprises controlling an intracellular zinc ion concentration in vitro.

47.-56. (canceled)

57. A method of screening for a compound having a degranulation reaction-controlling action, which comprises measuring an intracellular zinc ion concentration.

58. The screening method according to claim 57, wherein the cell is at least one selected from the group consisting of a neutrophil, an eosinophil, a basophil, a mast cell, a natural killer cell, a natural killer T cell, a cytotoxic T cell and a platelet.

59. The screening method according to claim 58, wherein the cell is a mast cell.

60. A method of screening for a compound having a cytokine production-controlling action, which comprises measuring an intracellular zinc ion concentration.

61. The screening method according to claim 60, wherein the cell is at least one selected from the group consisting of a neutrophil, an eosinophil, a basophil, a mast cell, a natural killer cell, a natural killer T cell, a cytotoxic T cell and a platelet.

62. The screening method according to claim 61, wherein the cell is a mast cell.

63. A method of controlling a degranulation reaction, which comprises administrating an effective amount of a substance capable of controlling an intracellular zinc ion concentration.

64. (canceled)

65. A method of controlling a cytokine production, which comprises administrating an effective amount of a substance capable of controlling an intracellular zinc ion concentration.

66. (canceled)

67. A method for the prophylaxis and/or treatment of an allergic disease and/or an inflammatory disease including an autoimmune disease, which comprises administrating an effective amount of a substance capable of controlling an intracellular zinc ion concentration.

68. (canceled)
